(19) United States
(12) Patent Application Publication
(10) Pub. No.: US 2019/0175156 A1
(43) Pub. Date: Jun. 13, 2019

(54) NOVELTY IN BIOPSY VALVE CAP HOLE EMBODIMENT

(71) Applicant: PERGO MEDIKAL VE ILAC SANAYI A.S., Izmir (TR)

(72) Inventors: Sevket Ahmet TAYGUR, Izmir (TR); Mahmut Melih KAVURKA, Izmir (TR)

(21) Appl. No.: 16/211,603

(22) Filed: Dec. 6, 2018

(30) Foreign Application Priority Data
Dec. 7, 2017 (TR) 2017/19856

(51) Int. Cl. A61B 10/04; A61B 10/06; A61B 17/12

(52) U.S. Cl. CPC A61B 10/04; A61B 10/06; A61B 17/3423; A61B 2010/045; A61B 17/12013

(57) ABSTRACT

A biopsy valve and valve cap are provided having a geometry for preventing unintended leakage of internal fluids during the biopsy procedure. The opening through which the forceps instrument is inserted is of a flexible structure and wraps the body of the forceps instrument without leaving any space, preventing leakage. The opening may have the same geometry as that of the forceps instrument.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

### 1. Field of the Invention

The present invention relates to a biopsy valve and the valve cap of the said valve.

### 2. Description of Related Art Including Information Disclosed Under 37 CFR 1.97 and 37 CFR 1.98

Endoscopy means the operation of examining the digestive system organs by use of an instrument having a tube shape which has a specific light source, provides for taking images by help of a camera located at the tip, and is twistable. The instrument is called an endoscope. A considerably soft tube of 8-10-millimeter thickness is directed and accessed to the stomach and duodenum through the esophagus. In colonoscopy, the entire colon is examined. If required, a biopsy is made for diagnosis or treatment operations such as stopping bleeding and removing of polyps.

Removing a tissue section from a living being in order to examine under microscope is called biopsy. Biopsy is conducted for patients who cannot be diagnosed during examination or similar examinations. The most frequently used method is a needle biopsy. A thin needle is inserted into the desired tissue location and a small part is extracted from such location by suction or dissecting.

An operation called closed biopsy is an operation for taking tissue from target organs by a needle and is less invasive. The endoscopy instrument used for biopsy operation has a component with a cap structure called a valve in the area where an instrument of tube shape called forceps makes entrance. The valve body and cap section have spaces for allowing entrance of the forceps. As seen from FIG. 3 showing the related art, the space in the valve cap is of a slot form. The forceps space in slot form in the related art cannot wrap the outer diameter of forceps entirely during insertion. Because of this, fluid exits from the open parts of the slot as seen in FIG. 4, during removal of forceps in particular. This outflow of fluid is entirely uncontrolled and in some cases it may jump to the height of up to one meter. Splashing of such bodily fluids adversely influences the working environment and may result in undesired consequences.

Another disadvantage of such embodiments is that the fluid applying pressure onto the cap to open because of internal pressure after closing the cap, and that the cap has the potential to open. Although there are steps providing grasping between the valve body and cap in current embodiments, the pressure exceeding a certain quantity will force the cap to open and cause negative influence on the operation.

Among the examples that can be given for the related art is the patent application numbered US20140187866, entitled “BIOPSY INLET VALVE” which discloses a space in slot form clearly on said valve cap. Furthermore, the cross-section figure shows that the cap and body overlaps in a flat surface and thus provides interlocking.

U.S. Pat. No. 6,165,124 discloses that the cap area of the forceps instrument where it makes entry should have a round geometry but an intermediate apparatus is used on the cap to provide such round geometry. In other words, a circular cross-sectional tube is located in the space of slit shape before the entry of the forceps instrument. Then the forceps instrument makes entry passing through this tube shaped apparatus. Thus, the forceps instrument is wrapped by a circular cross-sectional area. The necessity to use an intermediate mounting apparatus is the disadvantage aspect of this device.

### BRIEF SUMMARY OF THE INVENTION

From the current status of the related art, the purpose of the present invention is to prevent leaking and splashing of inter-body fluids from the valve component during removal of forceps instrument from endoscope device used in biopsy operations.

Another purpose of the invention is to provide a stronger cap and body connection against compressing force caused by interior pressure after closing the cap of the valve onto the body of the valve.

### BRIEF DESCRIPTION OF THE SEVERAL VIEWS OF THE DRAWINGS

FIG. 1 is a perspective view of the valve of the present invention in the open position.

FIG. 2 is perspective view of the valve of the present invention and the forceps instrument passing through the cap.

FIG. 3 is a perspective view of a biopsy valve of the prior art.

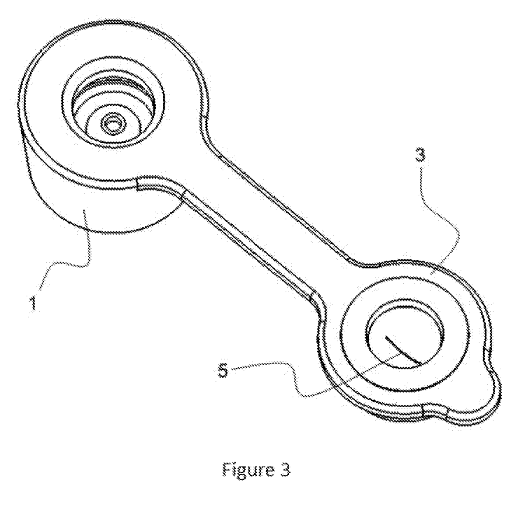

FIG. 4 is perspective view of the valve of the present invention and forceps instrument passing through the cap in the prior art.

FIG. 5 is side cross-section of the valve of the present invention in position showing combination with the body after closing the cap.

### REFERENCE NUMBERS

1. Body
1.1 Body tab
2. Connection cord
3. Cap
3.1 Cap tab
4. Hole
5. Slot
6. Forceps instrument
10. Valve

### DETAILED DESCRIPTION OF THE INVENTION

The detailed description of the invention discloses cases where the valve (10) used in the part wherein the forceps instrument (6) enters the body, provides prevention of outgoing bodily fluids during operation when endoscopy devices are used.

The invention relates to valves (10) containing a body (1) located on the entrance point during entrance of the forceps instrument (6) into device in endoscopy devices and a cap (3) closing onto the body (1), and it is characterized in that it comprises a hole (4), in the closed position not making air or fluid transfer between internal and external environment of the cap (3) and the body (1) before and after use, easily opening by help of a flexible structure when the very tip of said forceps instrument (6) contacts said cap (3) and wrapping the body of said forceps instrument (6) without leaving any space, going into the former closed position when said forceps instrument (6) is removed after use, located on the said cap (3) having the same geometric form as said forceps instrument (6).

As stated in the related art section, the space provided for passing of the forceps instrument (6) through particularly the cap (3) part of the valve (10) was in the form of a slot and splash of bodily fluids during removal of the forceps instrument (6) could not be prevented.

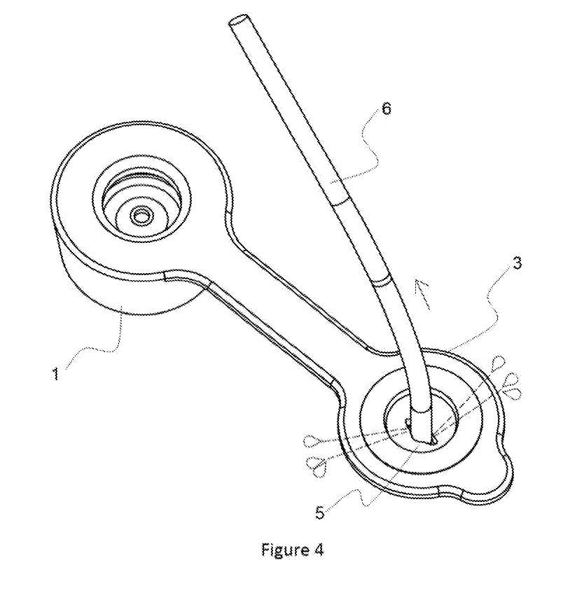

FIG. 4 illustrates the best pictures of this case.

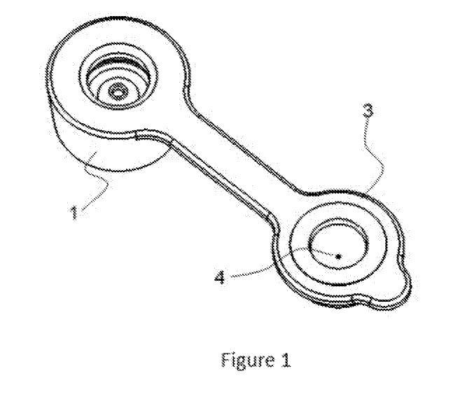

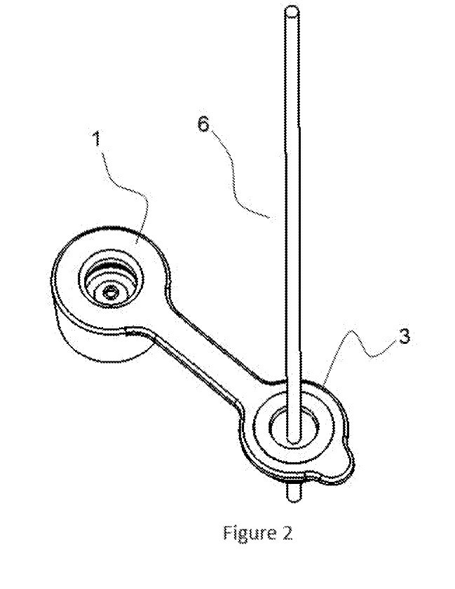

The picture illustrated in FIG. 1 of the new design of the present invention shows that the hole (4) in the cap (3) part of the valve (10) is a spot. In a better expression, said hole (4) is of the same form as the forceps instrument (6). Thus as seen in FIG. 2, during passing of the forceps instrument (6) through the cap (3) hole (4) and use thereof, the hole (4) wraps the outer diameter of the forceps instrument (6) entirely and thus prevents any likely leakage.

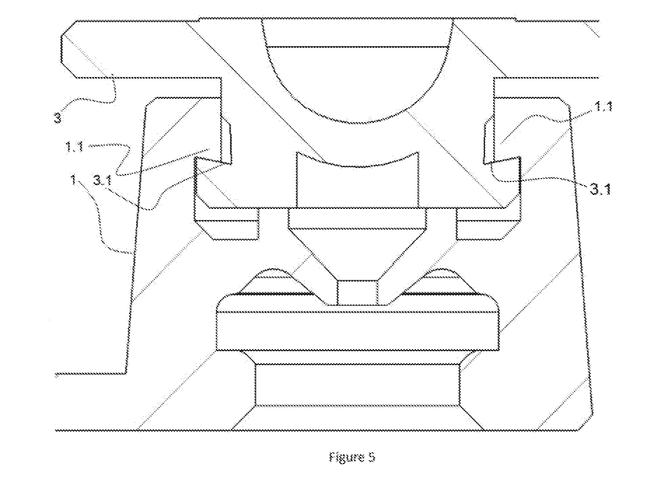

Another feature of the valve (10) having new the design is that consistent continuity of combination of the cap (3) and the body (1) is provided against fluid pressure from inside the body after the cap (3) is closed. As seen in FIG. 5, combination of the cap (3) with the body (1) is achieved by means of entrance of it there into. Two factors are seen in this process. They are the body tab (1.1) designed in the form of an extension inwardly and the cap tab (3.1) designed to extend outward. Said tabs are interlocked after the cap (3) seats into the body (1). The disadvantage of such locking encountered in the prior art is that both tabs are designed parallel to the surface. When FIG. 5 is examined carefully, it is likely to state that both tabs are designed at a certain angle with the surface and thus the cap (3) grasps the body (1) better against the fluid pressure coming through the body (1).

In conclusion, said disadvantageous cases are eliminated by means of this design having the valves (10) used in the endoscopy devices, which prevent leakage of bodily fluids and provide better combination of the cap (3) and the body (1) during use of valves (10) in endoscopy instruments.

In alternative embodiments of the invention, said hole (4) may have circular, triangle, square, rectangle, polygon or asymmetric form in such way to be of the same form as the forceps instrument (6).

I claim:

1. A valve containing a body located on the entrance point during entrance of forceps instrument into device in endoscopy devices and a cap closing onto the body and it is characterized in that it comprises of a hole, in the closed position not making air or fluid transfer between internal and external environment of the cap and the body before and after use, easily opening by help of flexible structure when very tip of said forceps instrument is contacted onto said cap and wrapping the body of said forceps instrument without leaving any space, going into former closed position when said forceps instrument is removed after use, located on the said cap having the same geometric form as said forceps instrument.

2. The valve according to claim 1, characterized in that it comprises of a cap tab formed in a manner the cap makes a certain angle with surface in order to enhance the strength against in-body fluid pressure provided by the combination after the valve cap closes onto the body.

3. The valve according to claim 2, characterized in that it comprises of a body tab having a cap tab of said angular geometry and providing parallel form with cap tab.

4. A valve according to claim 1, characterized in that said hole is circular in a manner to be in the same geometric form as the forceps instrument.

5. A valve according to claim 1, characterized in that said hole is triangle in a manner to be in the same geometric form as the forceps instrument.

6. A valve according to claim 1, characterized in that said hole is square in a manner to be in the same geometric form as the forceps instrument.

7. A valve according to claim 1, characterized in that said hole is rectangle in a manner to be in same geometric form as the forceps instrument.

8. A valve according to claim 1, characterized in that said hole is polygon in a manner to be in the same geometric form as the forceps instrument.

9. A valve according to claim 1, characterized in that said hole has an asymmetric form in a manner to be in same geometric form as the forceps instrument.
